(19) United States
(12) Patent Application Publication
(10) Pub. No.: US 2008/0233607 A1
(43) Pub. Date: Sep. 25, 2008

(54) Cell Culture Device

(76) Inventors: Hanry Yu, Singapore (SG); Yi-Chin Toh, Singapore (SG); San San Susanne Ng, Singapore (SG)

Correspondence Address: KLARQUIST SPARKMAN, LLP, 121 SW SALMON STREET, SUITE 1600, PORTLAND, OR, 97204, US

(21) Appl. No.: 11/667,715

(22) PCT Filed: Nov. 10, 2005
(86) PCT No.: PCT/SG05/00385

§ 371 Date: Jan. 7, 2008

Related U.S. Application Data
(60) Provisional application No. 60/626,963, filed on Nov. 11, 2004.

(51) Int. Cl. C12Q 1/20; C12M 1/00; G03F 7/20; C12N 5/00

(52) U.S. Cl. 435/29; 435/299.1; 430/322; 435/347

(57) ABSTRACT

The invention provides cell culture devices comprising a channel, the channel comprising one or more inlets and one or more outlets, and a cell retention chamber defined by an internal surface of the channel and a plurality of projections extending therefrom. The invention further provides methods of use relating to such cell culture devices.

### CROSS-REFERENCE TO RELATED APPLICATIONS

This application claims the benefit of U.S. Provisional Patent Application No. 60/626,963, filed Nov. 11, 2004, which is incorporated herein by reference.

### FIELD OF THE INVENTION

The invention concerns a cell culture device for the functional maintenance of cells, particularly anchorage-dependent cells, a method of making such a device and the use of such a device.

### BACKGROUND OF THE INVENTION

A strategy for the functional maintenance of anchorage dependent cells in vitro that have high fidelity in vivo is important and relevant to tissue engineering applications, development of pathological models and understanding the effects and mechanisms of potential therapeutic agents.

The maintenance of the liver specific functions of anchorage-dependent cells such as hepatocytes in vitro is useful for applications that employ primary hepatocyte models such as drug screening studies and bioartificial liver assisted devices (BLAD).

However, current primary hepatocyte models suffer from rapid loss of the liver specific phenotype within days in culture. The functional deterioration of hepatocytes in vitro has been attributed to the deficiencies of their culture environment to provide appropriate conditions that mimic an in vivo microenvironment that is highly organized both architecturally and compositionally.

Extensive research has been directed to identifying the various factors that enable the long-term maintenance of primary hepatocyte functions in vitro. Parameters that are typically considered in the long-term culture of primary hepatocytes are as follows:

3D Microenvironment

In vivo, hepatocytes, for example, are supported three dimensionally by a combination of extracellular matrix and other hepatocytes. It is known that the coating of two dimensional substrates with different matrix components show that although the provision of these substrates help hepatocytes live longer they do not significantly delay the onset of hepatocyte de-differentiation.

Fluid Flow

Fluid perfusion mimics the hepatic circulation, permitting an efficient, continuous transport of gas and nutrients to the hepatocyte and allows adequate removal of metabolic waste. Oxygen, in particular, is an important modulator of hepatocyte function and has been deemed as one of the primary regulators of the zonal variations in metabolism and detoxification between the periportal and perivenous regions of the liver. Therefore, hepatocytes have been shown to retain their functions better under dynamic culture as compared to static culture. It has been shown that although an increase flow rate is beneficial for the maintenance of hepatocyte functions by increasing the delivery of oxygen to the hepatocyte, excess shear stress induced by a higher fluid flow rate is detrimental to the hepatocyte functions.

Co-Cultures with Non-Parenchymal Cells

Co-cultures of hepatocytes with both liver derived and non-liver derived non-parenchymal cells (NPCs) such as biliary epithelial cells, sinusoidal and vascular endothelial cells, fibroblasts and stellate cells have been shown to enhance many liver specific functions. NPCs have also been postulated to enhance hepatocyte functions by secreting basement membrane components.

Establishment of Hepatocyte Polarity

The maintenance of differentiated functions of epithelial cells is strictly dependent on the establishment of morphological polarity. Hepatocytes, like other epithelial cells, are structurally and functionally polarized. The metabolic functions of hepatocytes have been positively correlated to the polarity of hepatocytes induced by different culture configurations. Accordingly, the recovery of hepatocyte polarity may be important in the maintenance of hepatocyte function.

Different culture models have been proposed for the long term culture of primary hepatocytes, each incorporating various degrees of the features discussed above in its design. The main configurations of primary hepatocyte culture models are as follows:

Sandwich Culture

This typically comprises a monolayer of hepatocytes sandwiched between two layers of a simple or complex matrix such as collagen or Matrigel® (a laminin rich matrix). This culture configuration has been shown to significantly augment hepatocyte function. When maintained in sandwich cultures, hepatocytes aggregate into cord-like structures and retain their phenotypic globular morphology.

Spheroids

Hepatocytes self-assemble into spheroids which are 3D organoids possessing tight junctions and microvilli-lined channels that resemble bile canaliculi. The enhancement of hepatocyte functions in spheroid cultures is mostly attributed to the secretion of a basement membrane lining the outside of the spheroid and the presence of homotypic and heterotypic cell-to-cell interactions and the reestablishment of polarity. Spheroids are formed by culturing hepatocytes alone or with other non-parenchymal cells on moderately adhesive surfaces or in suspension so as to induce hepatocyte aggregation to provide anchorage for the hepatocytes.

Bioreactor Based Systems

Hitherto, most current bioreactor systems have been developed for bioartificial liver assisted devices (BLAD). The main advantage of most bioreactor designs is that they allow for the simulation of the hepatic circulation to enhance oxygen and nutrient mass transfer for maintenance of hepatocyte function. Some of these bioreactor conceptual designs have been incorporated into in vitro models for drug biotransformation studies. These bioreactor systems typically involve the embedding of the hepatocyte mono-culture or co-culture in a matrix such as collagen and the cell matrix construct is then housed in hollow fibres or on flat plates where they can be perfused. Some bio-reactor systems use scaffolds as a support for the hepatocyte mono-culture or co-culture and the cell scaffold construct is perfused or dynamically cultured.

Microfabrication Based Systems

Microfabrication techniques allow a finer degree of control over the cellular phenotypes by manipulating cues in the local cellular environment. Homotypic and heterotypic cell-to-cell interactions between hepatocytes and fibroblasts can be controlled using photolithography methods in order to pattern the two cell types to modulate hepatocyte functions.

In addition, the numerous approaches to in vitro hepatocyte culture also include the following:

U.S. Pat. No. 5,624,840 discloses a three dimensional cell and tissue culture system for the long term culture of liver cells and tissues in vitro in an environment that more closely approximates that found in vivo. Here, the growth of stromal cells in three dimensions is used to sustain active proliferation of parenchymal cells in culture for longer periods of time than conventional monolayer systems.

U.S. Pat. No. 5,270,192 discloses a hepatocyte bio-reactor or bioartificial liver comprising a containment vessel having a perfusion inlet and a perfusion outlet. A matrix is provided within the containment vessel such as to entrap hepatocyte aggregates within the containment vessel while allowing perfusion of the matrix. The matrix is comprised of glass beads in the substantial absence of connective tissue.

U.S. 2002/0182241 A1 discloses scaffold structures that interconnect to build up a full, vascularized organ. Alternatively, the scaffolds can be formed by rolling or folding templates to form thick three-dimensional constructs. The scaffolds in this case serve as the template for cell adhesion and growth by cells that are added to scaffolds through the vessels, holes or pores of such scaffolds. A second set of cells, such as endothelial cells, can also be added to or seeded onto the scaffold. Once the sets of cells have been added to or seeded onto the three dimensional scaffold, this tissue engineered organ is implanted into a recipient.

The applicants have found that none of the above systems or current models are suitable for the long term culture of hepatocytes in vitro, especially for studies regarding pharmaceutical compounds and biological studies with respect to cell biology.

### SUMMARY OF THE INVENTION

The invention provides a cell culture device comprising a channel, the channel having one or more inlets and one or more outlets the channel comprising a cell retention chamber defined by an internal surface of the channel and a plurality of projections extending therefrom.

The present invention in a further aspect provides a method of making a cell culture device which method comprises the steps of:
(a) fabricating a mould using photolithography, and
(b) replicate moulding using a polymeric compound.

In still a further aspect, the invention provides method of culturing cells in the cell culture device as described herein, the method comprising the steps of:
(a) introducing one or more-types of cells suspended in methylated collagen into the cell retention chamber of the cell culture device; and
(b) introducing a terpolymer solution to initiate a complex coacervation reaction which results in gradual gelation of the collagen matrix.

The present invention provides in a further aspect a method for observing a cell culture in a cell culture device for bioimaging comprising:
(a) seeding the cell culture device with one or more cell types in a collagen matrix, and
(b) observing the one or more cell types with an imaging device.

The invention further provides a method of screening a plurality of candidate pharmaceutical compounds against a target comprising:
(a) seeding a plurality of cell culture devices with one or more cell types containing the target in a collagen matrix;
(b) perfusing the cell culture device with the candidate pharmaceutical compound in a fluid medium, and
(c) screening the cell culture devices to identify the desired pharmaceutical compound.

The present invention in a yet further aspect provides a method for the purification of a biological fluid comprising:
(a) seeding a plurality of cell culture devices with one or more cell types in culture matrix;
(b) perfusing the cell culture devices with the biological fluid, and
(c) obtaining the purified biological fluid.

The invention further provides a method comprising culturing cells in the cell culture device as described herein.

### BRIEF DESCRIPTION OF THE FIGURES

FIGS. 1A and 1B depict a plan view of a cell culture device in accordance with the present invention;

Formal 2A and 2B are perspective views of a cell culture device according to the invention;

FIG. 3 depicts hepatocytes embedded in a collagen matrix within the cell retention chamber of a cell culture device of FIGS. 1A and 1B;

FIG. 4 is a further perspective view of a cell culture device according to the invention;

FIGS. 5A, 5B, 6A and 6B show various ways to accomplish laminar flow and coacervation of collagen;

FIG. 7A depicts a first configuration of sinusoidal endothelial cells (SECs) that have been dynamically seeded in the cell culture device;

FIG. 7B depicts a second configuration of SECs that have been seeded by the complex coacervation of collagen and terpolymer;

FIG. 8 depicts a closed loop perfusion system for use with the cell culture device of FIGS. 1A and 1B; and

FIG. 9 depicts a closed loop perfusion system for use with microchannel devices;

In the figures like numerals denote like parts.

### DETAILED DESCRIPTION OF THE INVENTION

Cell Types

Cells may be isolated from any suitable animal. Preferably, they are isolated from mammals. Cells may include anchorage-dependent cells, such as hepatocytes, fibroblasts, bone marrow stromal cells and endothelial cells, chondrocytes, osteoblasts, myocytes, neural cells, and stellate cells. Hepatocytes may be isolated from rats of the Wistar type via, for example, two step collagenase perfusion such as that according to Chia et al., 2000. Sinusoidal liver endothelial cells (SECs) may be isolated, for example, according to Baret, 1994 using a Percoll® gradient.

Cell Culture Device

Microfluidic systems, such as the cell culture device of the present invention, have distinctive properties due to their small dimensions. One of them is that fluid flow in the cell culture device is laminar. Operating under laminar flow allows two or more layers of different fluids to flow next to each other without mixing other than diffusion of their constituent components across the interface.

The cell culture device in accordance with the present invention may generally be fabricated by photolithography methods, for example,.soft photolithography. Typically, soft photolithography may involve the following steps:
(a) fabricating a master mould using, for example, photolithography; and
(b) replica moulding with a polymeric compound using the master mould.

It will be appreciated that photolithography techniques are known to those skilled in the art.

Typically, the fabricating step comprises spin coating a wafer, which may be of, for example, glass or silicon, with a photoresist compound. The photoresist compound may preferably be of the negative high aspect ratio type. The photo resist compound may preferably be SU-8 by MicroChem Corp.

The spin-coated wafer may be masked in order to generate a pattern upon illumination with a light source. The spin coated wafer is typically illuminated with a light source, preferably, for example, ultraviolet light to generate a photo resist pattern. The photo resist pattern is then developed. The developed pattern may be used as a master mould in a subsequent replica moulding step.

A replica mould may be produced using the master mould. Typically, the replica mould may be manufactured from a siloxane containing polymer or any them oplastics, preferably, polydimetholsiloxane. It will be appreciated that other polymers, with varying desired properties can be used depending on the end application. For example, a radio-opaque material or a biodegradable material may be used.

The replica mould is preferably supported on a substrate. The substrate may, for example, comprise a glass or plastics material.

The replica mould may optionally be bonded to a glass substrate, such as a glass substrate, by, for example, oxidation in oxygen plasma.

Poly(dimethylsiloxane) (PDMS; sylgard 184, Dow-Corning) cell culture devices with a plurality of projections, may be fabricated by replica moulding on an SU8 master mould, which is patterned by standard photolithography. The design of the cell culture device may be generated by AutoCAD® 2005 and printed with a high-resolution plot (Innovative Laser System, Singapore). SU-8 high aspect ratio negative photoresist may be spih coated onto a second wafer (e.g., 500 rpm at 100 rpm/s, for 10 seconds and then 3000 rpm at 250 rpm/s for 30 seconds) and soft-baked at, for example, 95° C. for 1 hour. This is then followed by, for example, exposing for approximately 70 seconds, post-baking at 50° C. for 10 minutes and then at 95° C. for 30 minutes and developing for 30 minutes. A liquid PDMS prepolymer (e.g., 1:10 base polymer:curing agent) may then poured onto the master mould and cured, such as at 65° C. overnight before peeling off. The PDMS membrane may then optionally be oxidised in oxygen plasma for 1 minute (˜400 millitor) to chemically bond the membrane to a glass substrate.

A closed loop perfusion apparatus as shown in FIG. 9 may comprise a cell culture device 100 comprising one or more cell cultures in a three-dimensional collagen matrix. The cell culture device is located on a heating plate i to maintain the device at 37° C.

The cell culture device may be attached, at its inlets to three syringe pumps 2, 3, 4. Each pump 2, 3, 4 respectively contains culture medium, terpolymer or a suspension of cells in collagen. The pumps 2, 3, 4 will perfuse the cell culture device 1 with each of their respective solutions. Prior to entering the device 100, bubbles may be removed from the culture medium using a bubble trap 5. Used solutions may be disposed of via outlet 7. The syringe pumps containing the terpolymer and cell culture medium are connected via a four-way valve 6.

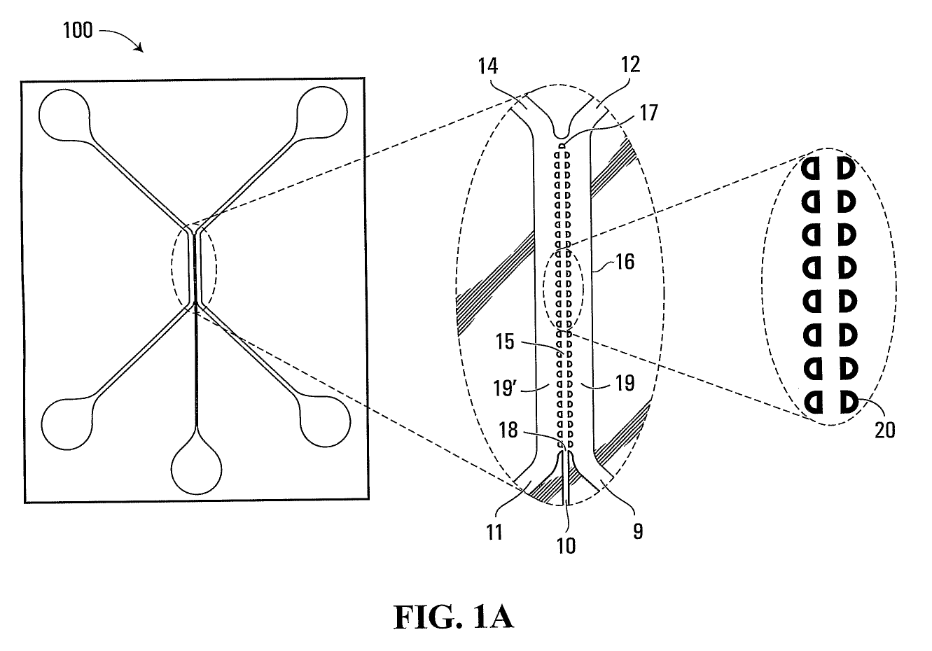

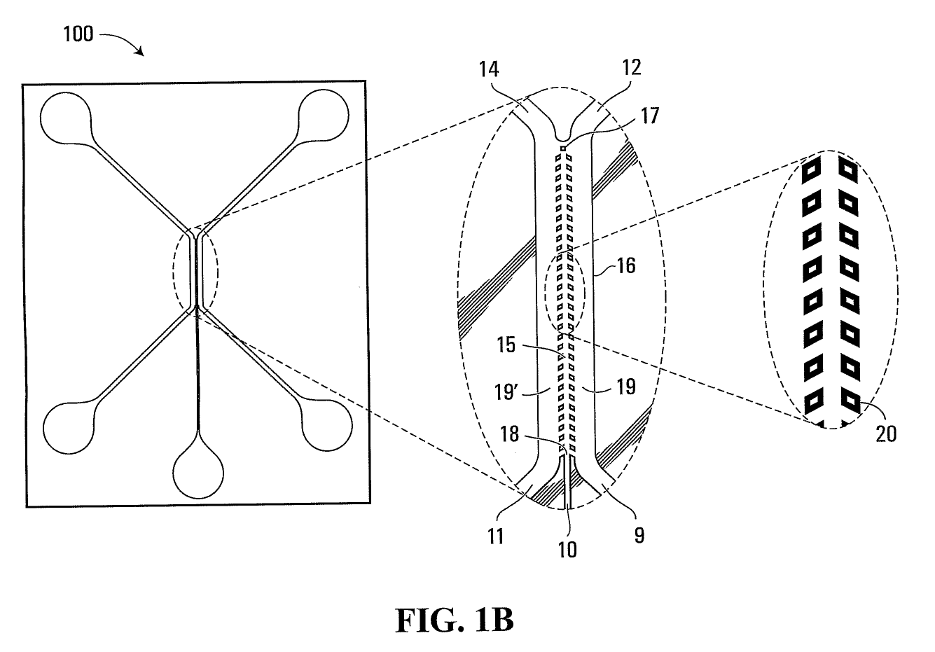

Referring to FIGS. 1A and 1B, a plan view of a cell culture device 100 in accordance with the invention is depicted. The device 100 may comprise a channel 16 having inlets 9, 10, 11 and outlets 12 and 14 and a cell retention chamber 15 defined by a plurality of projections 20 extending from an internal surface of the channel 16. The cell retention chamber 15 is closed to the passage of cells at an end 17 opposite to its opening 18. The cell culture device 100 also may be provided with a space 19, 19′ flanking the cell retention chamber to allow the perfusion of liquid media through the device. The perfused liquid media can exit the device via the outlets 12 and 14.

The projections 20 that define the cell retention chamber 15 may be spaced at least part of the way along the channel 16 at a gap distance which is smaller than the average diameter of a particular cell type, so as to trap cells, for example hepatocytes or SECS, within the cell retention chamber. Preferably, the projections may be arranged in two, spaced apart, substantially parallel rows, as shown in FIGS. 1A and 1B. In one embodiment, the projections 20 extend substantially upwardly from a bottom surface of the channel 23.

Preferably, projections are spaced apart at a gap distance of 1 to 20 μm, preferably 1 to 15 μm, more preferably 1 to 10 μm, most preferably 1 to 5 μm.

Projections with different dimensions and geometrical shapes, such as, circular, semi-circular, rectangular and square, may be used.

In one embodiment, the projections are rectangular in shape. The rectangular projections may be arranged at an angle relative to the plane perpendicular to the fluid flow path, preferably, in a chevron-like pattern. Rectangular projections may be positioned, for example, at an angle of between −90° to +90°, −45° to +45°, −20° to −25°, or +20° to +25°, most preferably at an angle of +22°, to the plane perpendicular to the path of fluid flow. Positive angles mean that the projections are angled such that, as shown in FIG. 1B, their inner edges are closer to the outlets 12 and 14 than their outer edges, i.e., the apex of the chevron is oriented towards the outlet end of the device.

The rectangular projections may be from 30 to 100 μm in width, preferably 60 to 100 μm, more preferably 70 to 100 μ, more preferably 80 to 100 μm, most preferably 90 to 100 μm in width.

The rectangular projections may be from 30 to 100 μm in length, preferably 60 to 100 μm, more preferably 70 to 100 μm, more preferably 80 to 100 μm, most preferably 90 to 100 μm in length.

The rectangular projections may be from 10 to 300 μm in height.

In one embodiment, the rectangular projections are 30 μm length and 50 μm in width.

In embodiments which include circular or semi-circular projections, the projections may be from 20 to 60 μm in diameter, preferably 30 to 50 μm, more preferably 40 to 50 μm in diameter. The projections may have a radius of from 20 to 40 μm. Preferably the radius may be 30 μm. Projections may be from 10 to 300 μm in height. In the most preferable embodiment, the projections have a radius of 30 μm, a diameter of 50 μm in diameter and a height of 50 μm.

In one embodiment, the cell culture device may further comprises a cell reservoir (not shown) connected to the channel. The cell reservoir can optionally be left open, so as to maximise fluid flow through the channel, or left closed, thereby minimising fluid flow through the channel.

Closed-Loop Perfusion Apparatus

The cell culture device (or a plurality thereof) may be integrated into a closed-loop microfluidic perfusion apparatus.

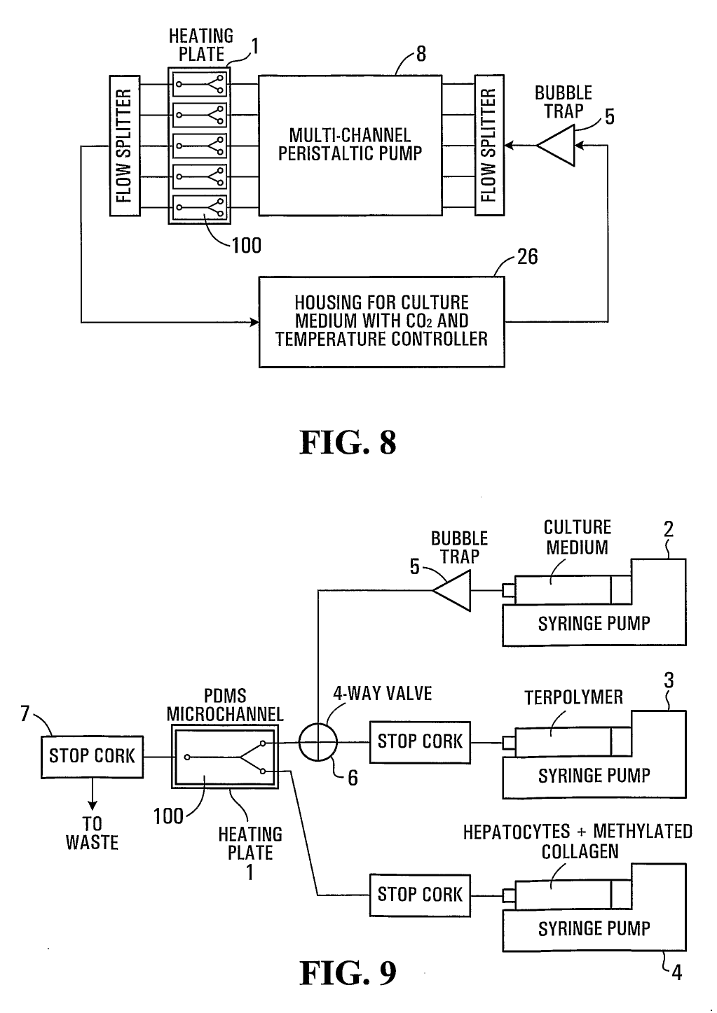

Referring to FIG. 8, the closed-loop apparatus comprises one or more cell culture devices 100, comprising one or more cell cultures in a three-dimensional collagen matrix, located on, means for heating, such as a heating plate 1, to maintain the cell culture devices 100 at, for example, 37° C. Other means for heating may include a water bath, an incubator or a microscope heating stage.

The cell culture devices 100 may be attached, at their inlets, to a pump 8, which may be, for example, a. peristaltic pump. The pump 8 can perfuse the cell culture devices 100 with culture medium. Prior to entering the peristaltic pump 8, bubbles are removed from the culture medium using a bubble trap 5.

The culture medium is located in a housing 26 where carbon dioxide and temperature can be maintained at, such as at 5% and 37° C., respectively.

The cell culture medium may be re-circulated back to the housing 26 upon its removal from the cell culture devices 100.

Incorporation of Collagen Matrix Support within the Cell Retention Chamber by the Complex Coacervation of Methlyated Collagen and Terpolymer Under Laminar Flow Conditions

A collagen matrix may be provided to support cells, such as, hepatocytes in a cell retention chamber of a cell culture device in accordance with the invention. The collagen matrix may be located within the cell retention chamber such that the collagen provides support for the cells but does not obstruct or occlude the perfusion of media through the device. The cells and collagen matrix may be introduced to the device in the form of a collagen-cell suspension in parallel with a terpolymer solution. The cells are trapped in the cell retention device and the collagen gel forms in situ via the complex coacervation reaction between the methlyated collagen and terpolymer under laminar flow conditions. Cell culture medium may replace the terpolymer during perfusion.

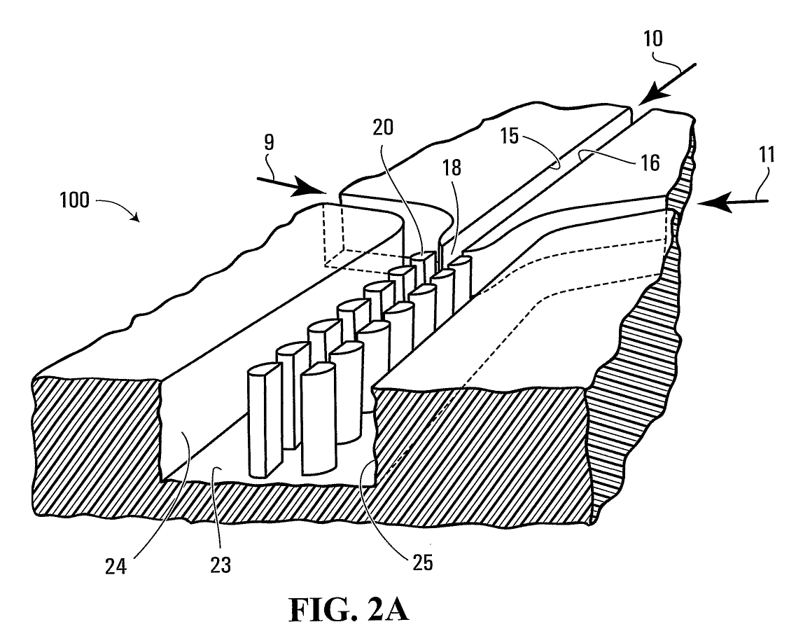

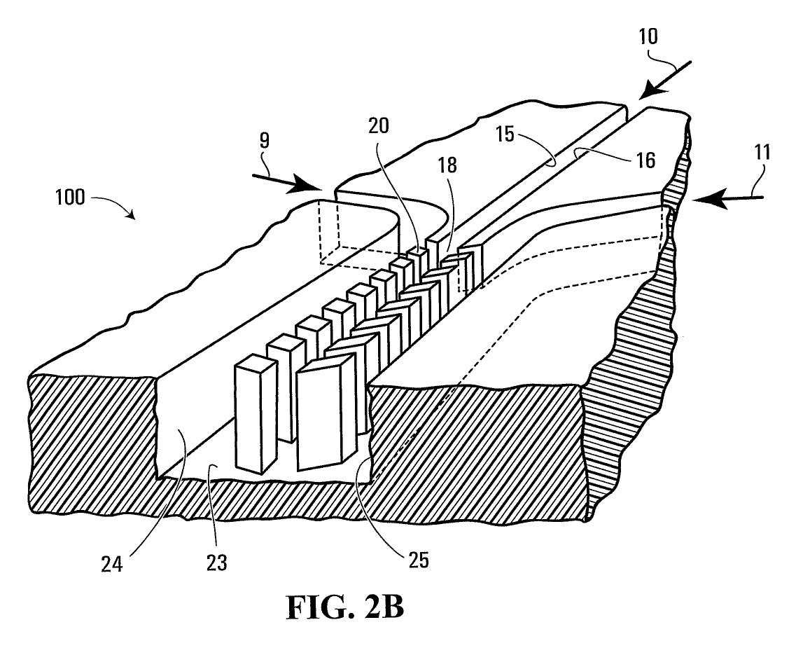

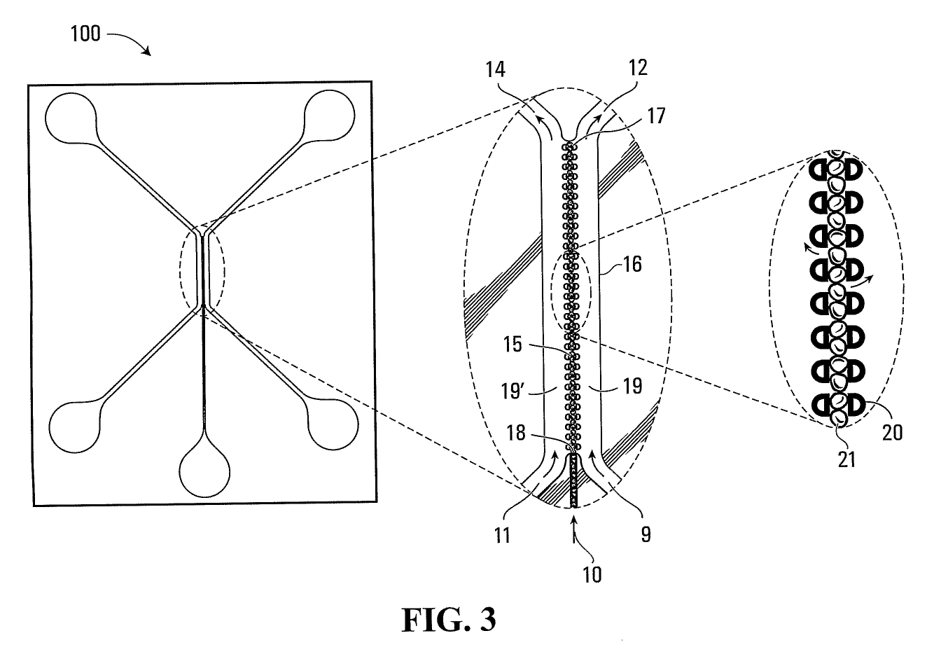

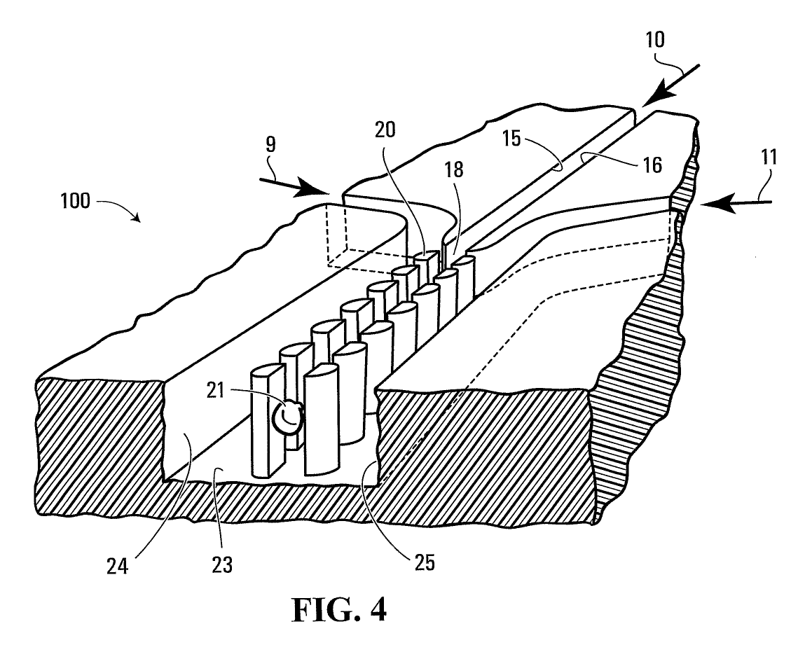

Referring to FIG. 3, hepatocytes 21 are shown in a collagen matrix in the cell retention chamber 15 of the cell culture device 100. The collagen matrix is within the cell retention chamber 15 so it does not obstruct or occlude the flanking spaces 19, 19′ either side of the cell retention chamber 15. Drawings are for illustration purposes only. Hepatocytes 21 may be present in the culture device, for example, in layers or aggregates.

Implementation of an Hepatocyte-SEC Co-Culture Model Wherein Hepatocytes and SECs are Spatially Localised in the Micro-Fluidic Channel

The strategy for spacially controlling the seeding of SECs may be classified into two categories:
Dynamic seeding
Entrapment by complex coacervation under laminar flow conditions

In the first strategy, hepatocytes may be three-dimensionally trapped in the cell retention chamber as described above. Subsequently SECs may be dynamically seeded such that they form a layer outside of the confinement of the hepatocytes. However the seeding of hepatocytes in this way is dependent on the SECs attachment to the collagen-terpolymer complex and PDMS projections. This can be improved by coating the projections with proteins derived from the extracellular matrix.

The second strategy involves the entrapment of SECs as a separate layer of the collagen gel in the cell retention chamber by using the complex coacervation of methylated collagen and terpolymer under laminar flow conditions.

FIGS. 7A and 7B schematically illustrate configurations for the spacially-localised seeding of SECs 22 in the cell culture device 100. Referring to FIG. 7A, an example of dynamic seeding is shown. Hepatocytes 21 may be physically confined to the cell retention chamber 15 after being introduced through inlet 10. Terpolymer is concommitantly introduced through inlet 9 and 11. SECs 22 are dynamically seeded externally of the cell retention chamber 15. Any liquid medium can exit via outlets 12, 13 and 14.

Referring to FIG. 7B, the entrapment of SECs 22 by laminar flow complex coacervation of methylated collagen and terpolymer is depicted. Hepatocytes 21 suspended in collagen, are introduced through inlet 11, SECs 22 suspended in collagen are introduced through inlet 10 and terpolymer is perfused through inlet 9 into the cell culture device 100 under laminar flow. Hepatocytes 21 are entrapped in the cell retention chamber 15 and SECs 22 are located externally of the cell retention chamber 15 but in contact with the PDMS projections by complex coacervation of collagen and terpolymer. Liquid medium exit via outlets 12, 13 and 14.

In both FIGS. 7A and 7B, hepatocytes 21 are shielded from shear force exerted by medium perfusing through the cell culture device 100 by a layer of SECs 22. This is similar to the physiological conditions in vivo. Drawings are for illustration purposes only. Hepatocytes 21 and SECs 22 may be present in the culture device in, for example, layers or aggregates.

Determination of Cell Number Within the Cell Culture Device

Hepatocytes 21 are fluorescently stained by incubating with, for example, 7-ethoxyresorufin for four hours prior to entrapment within the cell retention chamber 15. Images (e.g. 512 by 512 pixels) of an optical section spanning the height of the cell retention chamber 15 may be taken at an interval of two micrometers with a 20× objective lens. The images may be processed with Image Pro™ Plus to quantify the number of cells in the optical stack. The total number of cells in the cell retention chamber 15 can be estimated as the number of cells in cell retention chamber 15 is equivalent to the number of cells in the optical stack multiplied by the volume of cell retention chamber 15 divided by the volume of optical stack.

Assays

The metabolic functions of hepatocytes in the cell retention chamber 15 may be determined by using the 7-ethokyresorufin-O-de-ethylation assay (EROD) and 7-ethoxycoumarin-O-de-ethylation assay (ECOD) to determine the activities of CYP1A1 and CYP2B6 isozymes. Other metabolic functions may be evaluated based on urodine diphosphate glucoronosyltransferase (UGT) and sulphotransferase (ST) activities on the glucoronidation and sulphation of 7-hydroxy coumarin.

EROD Assay

The de-ethylation of ethoxy resorufin is CYP1A1 associated and its activity may be quantified under a confocal microscope according to Chiu et al., 2000. 7-ethoxyresorufin is perfused through the cell culture device 100, such as at 0.3 ml per hour for four hours. The cell culture 100 device may then visualized under a confocal microscope with a rhodamine filter. The images may then processed with Image Pros Plus to quantify the EROD activity.

ECOD Assay

The de-ethylation of 7-ethoxycoumarin is mediated mainly by CYP2B6 but can also be performed by several other forms of CYP enzyme, for example, 1A1/1A2/2A6 and 2E1. Different concentrations (20:150 μM) of 7-ethoxycoumarin may perfused through the cell culture device 100 at, for example, 0.3 ml per hour. To calculate the Michealis-Mentin kinetics, aliquots of the supernatant medium may be withdrawn after different periods of times to calculate the enzyme's time dependence. Samples are stored frozen, such as at −20° C., until analysis.

After thawing, 7-hydroxycoumarin conjugates may be cleaved using beta-glucuronidase in 100 U/ml acetate buffer overnight at 37° C. Aliquots of the treated samples may then be mixed with glycine buffer. The formation of 7-hydroxycoumarin may be quantified by fluorometry with an excitation wavelength of 360 nm and an emission wavelength of 460 nm. The spectrofluorometer is calibrated using 7-hydroxycoumarin standards.

UGT and ST Assays

Both enzyme activities may be measured in only one assay because both enzymes metabolize the substrate 7-hydroxycoumarin into 7-hydroxycoumarin glucoronide and 7-hydroxycoumarin sulphate. The detection of 7-hydroxycoumarin, 7-hydroxyglucoronide and 7-hydroxycoumarin sulphate may be performed by capillary electrophoresis according to Duffy et al., 1998. Separation may be carried out on untreated fused silica capillary with detection at 320 nm. Different concentrations of 7-hydroxycoumarin dissolved in Krebs-Hanseleit buffer may be perfused through the cell culture device, such as at 0.3 ml/hr, to calculate the Michealis-Menten kinetics. Aliquots of the supernatant medium can be withdrawn after different periods of time to investigate the enzymes' time dependence. 7-hydroxycoumarin standards may be prepared from a 1 mg/ml stock solution prepared in ethanol and ultrapure water (10:90 v/v). Both 7-hydroxycoumarin glucuronide and 7-hydroxycoumarin sulphate standards may be prepared from a 1 mg/ml stock prepared in ultra pure water. All standards are diluted with Krebs-Hanseleit buffer.

Cell Cultures and Extracellular Matrix Support

In use, the cell culture device 100 in accordance with the present invention may comprise one or more cell cultures located in the cell retention chamber 15. The one or more cell cultures may be introduced into the cell retention chamber 15 via the one or more inlets of the cell retention chamber 15. The cell cultures are preferably introduced to the cell retention chamber 15 in a liquid carrier. The liquid carrier may be cell culture medium.

Preferably, the one or more cell cultures are embedded in an extracellular matrix within the cell containment chamber. The extracellular matrix may comprise one or more proteins such as collagen, fibronectin, laminin, fabrillin, elastin, glycosaminoglycans, chitosan, alginate, or proteoglycans.

Preferably, the extracellular matrix in which the cells are embedded may be of the collagen type. More preferably, the collagen may be selected from the group consisting of collagen I, II, III, IV, V, VI, VII, VIII, IX, X, XI and XII Most preferably, the collagen may be collagen I.

The collagen may preferably be chemically modified. The chemical modification is preferably achieved by methylation or glycosylation, or a combination thereof. If the collagen is glycosylated it is preferably achieved by galactosylation.

The methylation of collagen may typically achieved by, for example, stirring precipitated collagen in acidified methanol.

The addition of galactose into collagen molecules may preferably be achieved by, for example, the reaction of collagen and 1-N-(lactobionic acyl)-ethylenediamine with the carboxyl group activator 1-ethyl-3,3′-dimethylaminoepropyl carbodiimide. The degree of collagen galactosylation may be quantified by a colourimetric method. Briefly, galactosylated collagen may be reacted with phenol and concentrated sulphuric acid, and the degree of colouration may be measured using a colourimeter at a wavelength of 510 nm using different concentrations of D-galactose BPS solutions as standards and unmodified collagen as a negative control.

Advantageously, the methylation and galactosylation of collagen reduces the density of collagen and the number of connections between collagen molecules. This allows increased perfusion of a cell culture embedded in a collagen matrix. Even more advantageously, an increase in collagen methylation is correlated with decreased densities and connections between collagen molecules.

In use, collagen, together with one or more cell cultures, is preferably introduced to the cell culture device 100 together with a terpolymer. The terpolymer may be, for example, HEMA-MMA-MAA. The collagen-cell mixture and terpolymer may be introduced separately, but concomitantly, into the cell culture device.

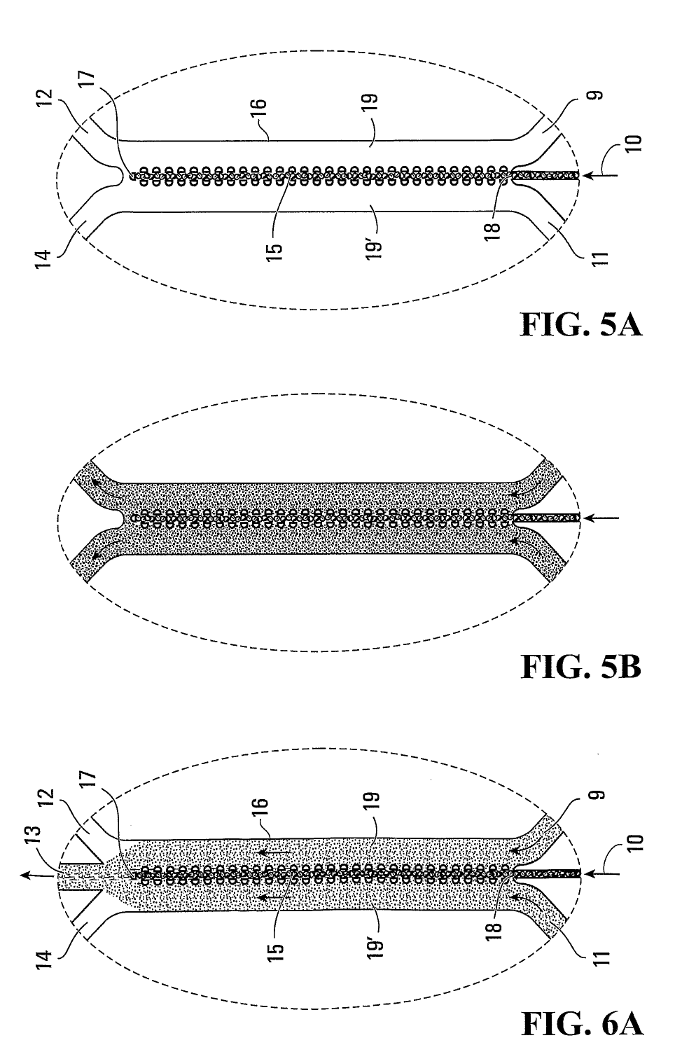

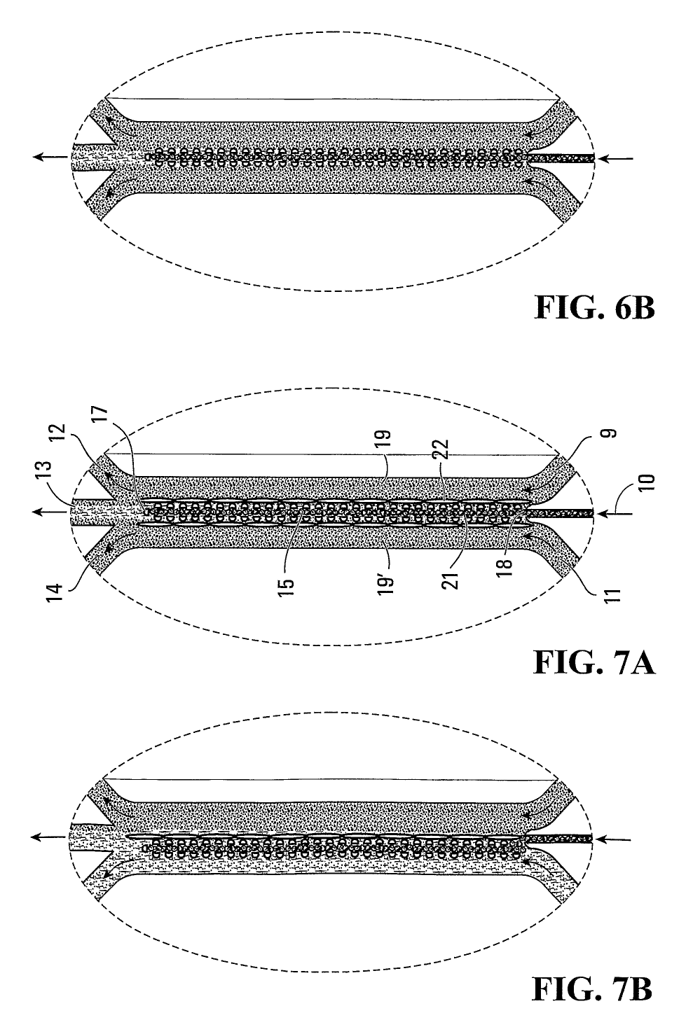

In one embodiment, by flowing two polyelectrolytes, in particular, collagen (containing one or more cell cultures) and HEMA-MMA-MAA into the cell culture device 100, the terpolymer solution is introduced into the spaces 19 and 19′ flanking the cell retention chamber 15. This allows the complex coacervation reaction between the cationic collagen and anionic terpolymer to result in the gradual gelation of the collagen which in turn traps the cell culture inside the cell retention chamber 15 in such a way that they are supported, three-dimensionally, by a collagen-based matrix (FIGS. 5 and 6).

In an embodiment, cells may be supported in three-dimensions by the collagen matrix for the preservation of the globular phenotype of hepatocytes which is correlated with maintenance of liver specific function.

The introduction of the collagen and terpolymer separately ensures that collagen and the terpolymer do not mix, thereby spatially constraining the cell culture to a portion of the cell culture device 100. This portion is preferably the cell retention chamber 15 or a portion thereof. In particular, the property of laminar flow within the cell culture device 100 ensures that when the collagen and/or cell culture and terpolymer are introduced into the cell culture device 100 there is substantially no mixing of the terpolymer and collagen/cell structure.

Typically, the terpolymer solution may be subsequently replaced with culture media to allow perfusion of the cells within the cell retention chamber 15.

Laminar flow provides for the seeding of two cell types in two discrete layers within the cell retention chamber in the substantial absence of mixing of the two cell types except at their respective interfaces.

The one or more cell cultures may be, for example, hepatocytes, fibroblasts, endothelial cells and bone marrow stromal cells, or other anchorage-dependent cells. In one embodiment, cell cultures may include, for example, CHO and HeLa cells.

Preferably, the one or more cell types comprises hepatocytes and endothelial cells.

The endothelial cells may be, for example, liver sinusoidal endothelial cells (SECs) 22. The liver sinusoidal endothelial cells may be introduced into the cell retention chamber 15 dynamically or by complex coacervation of collagen, premixed with SECs 22, and the terpolymer under laminar flow conditions.

The SECs 22 may be located, for example, on the projections 20 of the cell retention chamber 15, either internally therein or externally thereof. When the SECs 22 are located externally of the cell retention chamber 15 the projections 20 may preferably be coated with an extracellular matrix protein as defined in the group above (FIG. 7A).

In an embodiment, the invention provides two discrete layers of cells embedded in an extracellular matrix. Typically, this may be achieved by for example introducing to the cell retention chamber, by an inlet, a first cell culture, premixed with collagen or other extracellular matrix protein, in laminar flow with the terpolymer introduced to the device by another inlet. The collagen-cell mixture is allowed to set into a gel to form a first layer. A second cell culture (which may or may not be different from the first cell culture) also premixed with collagen or other extracellular matrix protein is introduced, by an inlet, to the cell retention chamber, in laminar flow with the terpolymer introduced into the cell culture device by another inlet.

In this embodiment, the first layer of cells is shielded by the upper layer of cells from any shear force generated by the perfusion of liquid medium through the cell culture device. This is similar to the in vivo milieu of the hepatocytes and endothelial cells.

Cell culture devices of the invention allow for the spacial control of cell seeding. In particular, the device allows emulation of the linear structure of hepatocytes in vivo. Moreover, the seeding of a second discrete layer of cells, for example NPCs, further emulates the in vivo physiology of the hepatocyte.

Uses

The cell culture device in accordance with the present invention may find application in complex tissue engineering, in particular, as an in vitro model of liver tissue. This application may be useful in xenobiotic toxicity studies in the liver and may be used in studies of liver-cancer and its mechanisms of metastasis.

The cell culture device may be used as a ‘biochip’ for biological imaging and other studies. The device may provide, for example, live imaging of cells and in particular, imaging of the dynamics of hepatocyte repolarisation and regeneration; protein trafficking and endocytosis and the like. The biological imaging may be used to characterise cell-to-cell interactions, cell-matrix interactions and the like.

The biochip may also be used in high-throughput screening to identify potential pharmaceutical compounds from a library of chemicals. The biochip may also, for example, be used to optimize delivery protocols of pharmaceutical agents, for example, concentration, volume, or frequency of delivery. This may be carried out using a plurality of cell culture devices in parallel for simultaneous monitoring of real-time effects.

The biochip may also be used to assay for toxicity of xenobiotics/pharmaceuticals and interactions (either advantageous or adverse) between pharmaceutical/xenobiotic compounds.

The cell culture device may also find application in the field of bio-artificial liver assist devices. These devices may comprise a plurality of cell culture devices which may serve as an intermediate form of treatment for a patient prior to having a liver transplant. Blood from a patient may, for example, be perfused through a cell culture device before returning to a patient's bloodstream in a similar way to the circulatory pathway of the liver.

The following examples are offered by way of illustration and not by way of limitation.

### EXAMPLE 1

Isolation of Cells

Hepatocytes were harvested from male Wistar rats weighing from 250 to 300 grams by a two step in situ collagenase perfusion method according to Chia et al., 2000. SECs were isolated according to Baret, 1994 using a Percoll® gradient in conjunction with selective attachment for separate SECs from Kupfer cells.

Characterisation of the Physical Properties of the Collagen Fibre Support

In order to reduce the density of the collagen matrix, collagen was subjected to chemical modification by a combination of methylation and galactosylation.

Collagen was methylated by stirring precipitated collagen in acidified methanol.

Characterisation of the degree of methylation was characterised by capillary electrophoresis. Capillary electrophoresis was carried out with 0.05% hydroxypropyl methylcellulose at a pH of 2.5 and a temperature of 21° C. This resolved the methylated collagen into four major peaks. An increase in the degree of methylation was correlated with an increase in the ratio of the areas under the last two peaks over the first two peaks, defined as Y. Collagen methylated at 4° C. for 6 days had a calculated Y value of 1.4, and was characterized as slightly methylated collagen (SM-collagen). Collagen methylated at 23° C. for 1 day had a calculated Y value of 1.9, and was characterized as highly methylated collagen (HM-collagen).

Galactose was incorporated into collagen by the reaction of collagen and 1-N-(lactobionic acyl)-ethylenediamine with the carboxyl-group activator 1-ethyl-3-3′-dimethylaminopropyl carbodiimide.

The degree of collagen galactosylation was quantified by a calorimetric method. Galactosylated collagen was reacted with phenol and concentrated sulphuric acid. The degree of coloration was then measured on a colorieter at a wavelength of 510 nm. A standard curve was plotted using varying concentrations of D-galactose in phosphate buffered saline to calculate the degree of galactosylation. Unmodified collagen was used as a negative control.

Performing the galactosylation reaction at 4° C. for 24 hours gave a galactosylation level of 80%. This level of galactosylation was used in subsequent studies.

The galactdsylated collagen was mixed with slightly methylated collagen and complex coacervated with terpolymer to provide an extracellular matrix support with variable physical and chemical properties. A decrease in the proportion of methylated collagen in the mixture of galactosylated and methylated collagen resulted in a decrease in collagen fibre density and connectivity.

In order to noninvasively characterise the formation of collagen nano-fibres in the extra-cellular microcapsule based three-dimensional microenvironment a back scattering confocal microscopy assay was used. An Olympus Fluoview® 500 confocal microscope was used with a 60× WLSM lens of NA 1.00. 2 μm sections of the microcapsule were obtained by optical sectioning for subsequent analysis. Three physical parameters were calculated using Image-Pro Plus 4.5.1 to describe the nano-fibre density (fractional area of dendrites=area of dendrites in pixels over the total pixels in the slice), nano-fibre length (mean dendritic length=average length of dendrites connected to a node per slice), and nano-fibre branching (mean dendrite number=average number of dendrites connected to a node per slice).

A summary of the physical characteristics of the microcapsule shown in Table 1 below.

TABLE 1

| | Normalised | | |
|---|---|---|---|
| | fractional | Normalised | Normalised |
| Modified | area of | dendritic | dendrite |
| Collagen | dendrites | length | number |
| SM-collagen | 1.000 ± 0.043 | 1.00 ± 0.12 | 1.00 ± 0.08 |
| HM-collagen | 0.502 ± 0.077 | 0.35 ± 0.16 | 0.38 ± 0.02 |
| % G-collagen | | | |
| in methylated | | | |
| collagen | | | |
| mixtures | | | |
| 17 | 0.964 ± 0.051 | 0.89 ± 0.06 | 0.95 ± 0.09 |
| 25 | 0.959 ± 0.053 | 0.74 ± 0.08 | 0.72 ± 0.15 |
| 50 | 0.952 ± 0.040 | 0.68 ± 0.17 | 0.66 ± 0.17 |
| 75 | 0.950 ± 0.036 | 0.64 ± 0.07 | 0.61 ± 0.07 |
| 83 | 0.929 ± 0.032 | 0.44 ± 0.11 | 0.58 ± 0.07 |

Table 1. Collagen nanofibre density, length and branching in a microcapsule were represented by the normalised fractional area of dendrites, dendritic length and dendrite number respectively. Values indicate normalised index ± standard deviation. SM-collagen: slightly methylated collagen; HM-collagen: highly methylated collagen; G-collagen: 80% galactosylated collagen.

Hepatocyte Culture in an Engineered Collagen Matrix

Primary rat hepatocytes seeded at an optimal density of 5×10⁶ cells/ml maintained the round phenotypic morphology of hepatocytes in a methylated collagen-terpolymer microcapsule. The hepatocytes were loosely supported by collagen nano-fibres in the microcapsule and showed enhanced differentiated functions over hepatocytes in monolayer culture.

Hepatocytes cultured within collagen matrices (1×10⁶ cells/200 μl) with varying physical and chemical properties demonstrated increased urea production when the physical support was increased (highly to slightly methylated collagen) and these functions could be further enhanced when the proportion of galactosylated collagen was increased.

Microfluidics-Based Delivery of Collagen

Microfluidic systems, such as the cell culture device of the present invention, have distinctive properties due to their small dimensions. One of them is that fluid flow in the cell culture device is laminar. Operating under laminar flow allows two or more layers of different fluids to flow next to each other without mixing other than diffusion of their constituent components across the interface.

1.5 mg/ml neutralised type I bovine dermal collagen was delivered into a cell culture device in accordance with the present invention. The architecture of the nanofibre matrix in the cell culture device was similar to that achieved by the pipetting technique used in collagen sandwich cultures.

Optimisation Three-Dimensional Entrapment of Cells in Cell Culture Devices Using Laminar Flow Complex Coacervation

Cell culture devices were fabricated as described previously. 6×10⁶ cells/ml of primary rat hepatocytes were suspended in 3.0 mg/ml of methylated collagen before being introduced into a closed loop perfusion apparatus as shown in FIG. 8.

The collagen-cell solution was pumped in parallel with 3% terpolymer solution. Upon formation of the collagen matrix, collagen flow was stopped and the terpolymer solution replaced with cell culture medium to perfuse the entrapped cells. Laminar flow inside the cell culture device ensured that the collagen and terpolymer did not mix thereby spacially constraining the cells to a portion of the cell culture device. The complex coacervation reaction between the cationic methylated collagen and anionic terpolymer resulted in the gradual gelation of the methylated collagen which trapped the cells in a three-dimensional matrix. Methylated collagen and terpolymer were prepared according to the method of Chiu et al., 2000.

Optimisation of Cell Number in the Cell Culture Device

Homotypic interactions between hepatocytes are vital for the maintenance of cell polarity and functionality. Accordingly, it is important that the three-dimensional entrapment of hepatocytes by laminar flow coacervation is able to load hepatocytes in the cell culture device at a density sufficient to achieve cell-to-cell interactions.

Different initial cell seeding densities were used to quantify the number of cells located in the cell culture device. Hepatocytes were fluorescently labelled by incubation with 7-ethoxyresorufin for 4 hours prior to loading of the cell culture device. Images (512×512 pixels) of an optical section spanning the height of the device (200 μm) were taken at an interval of 2 μm with a 20× objective lens using a confocal laser scanning microscope (Olympus Fluoview® 500). The images were then processed with Image-Pro® Plus to quantify the number of cells in the optical stack. An optical stack was taken at intervals along the cell culture device to see if there was any variation in the cell density along the length of the cell culture device.

It was observed that the number of cells in the cell culture device was low and was generally insensitive to the cell seeding density. Hepatocytes were also observed to flow out of the cell culture device even when the flow of the collagen-cell suspension was stopped.

When the initial cell seeding concentration was increased to greater than 6×10 cells/ml, the cells occluded the cell culture device and laminar flow complex coacervation could not be achieved.

Three-Dimensional Spacially Localised Entrapment of Hepatocytes and Fibroblasts in Cell Culture Devices by Using Laminar Flow Complex Coacervation

Cell culture devices with three inlets were fabricated by the moulding of PDMS (PDMS; sylgard 184, Dow-Corning) on a micromachined polycarbonate template. The PDMS membrane was then treated by oxygen plasma to chemically bond it to a glass substrate. 6×10⁶ cells/ml of primary rat hepatocytes or NIH 3T3 fibroblasts were suspended separately in 3.0 mg/ml of methylated collagen before being pumped into a closed loop perfusion apparatus as described above. The collagen-cell solution was pumped in parallel with 3% terpolymer solution. Hepatocytes and fibroblasts can be three-dimensionally entrapped in two discrete layers within the cell culture device.

### EXAMPLE 2

High Density Seeding of Hepatocytes in a Cell Culture Device

Different projection designs were evaluated based on their efficacy at cell entrapment within a cell culture device. The projection dimensions ranged from 30-50 μm and were of different geometrical shapes. Cell culture devices (100 μm (W)×100 μm (H)×1 cm (L)) with various projection designs were drawn using L-Edit (Tanner Research, Inc, USA) and translated into photomasks (Innovative Laser Systems, Singapore). A silicon master template was fabricated using standard deep reactive ion etching (DRIE) technology. A pre-polymer solution of poly-(dimethylsiloxane) (PDMS) (PDMS; Sylgard 184, Dow-Corning) was then poured over the template and cured at 65° C. overnight before being peeled off. The PDMS membrane was then oxidized in oxygen plasma for 1 minute (125 watts, 13.5 MHz, 50 sccm and 400 millitorr) for irreversible chemical bonding to glass coverslips. The cell culture devices with projections were then qualitatively evaluated for their cell entrapment efficacy by introducing hepatocytes suspended in 1× phosphate buffer saline (PBS) using a syringe pump into the cell culture device.

Dynamic Seeding of Hepatocytes Into Cell Culture Devices with Projections, and Assessment and Quantification of Cell Viability by Fluorescence Staining

Various methods to dynamically seed hepatocytes into the cell culture devices with projections were investigated to determine an acceptable operation window for the process. Hepatocytes were introduced into the cell culture device by either infusing or withdrawing a cell suspension (1.5×10⁶ cells/ml) from a syringe pump at different flow rates. The effect of different dynamic seeding parameters on hepatocytes' viability in the cell culture device was evaluated using fluorescence dyes, Cell Tracker Green (CTG) (Molecular Probes, Oregon) and Propidium Iodide (PI) (Molecular Probes, Oregon), to stain for live and necrotic cells, respectively.

The viability of hepatocytes after dynamic seeding into the cell culture device was assessed by fluorescence dyes, Cell Tracker Green (CTG) and Propidium Iodide (PI) (Molecular Probes, Oregon) to stain for live and necrotic cells respectively. The cell culture device was then perfused at 0.8 ml/hr with 20 μM of CTG diluted in culture medium (HepatoZYME-SFM (Invitrogen Corporation, Grand Island, N.Y.) supplemented with penicillin/streptomycin, dexamethasone and 60 mM HEPES (Invitrogen Corporation, Grand Island, N.Y.)) for 30 minutes, followed by culture medium for 30 minutes and finally 50 μg/ml of PI for 15 minutes. The cells were then fixed with 3.7% paraformaldehyde (PFA) for 30 minutes and viewed under a confocal laser scanning microscope (Olympus Fluoview 300). A quantification of the cell viability was performed by using image processing (Image-Pro® Plus 4.5.1, Media Cybernatics Inc., MD) to quantify the number of live and dead cells, and the percentage cell viability was normalized against static controls.

Results

The projection dimensions ranged from 30-50 μm and were of different geometrical shapes. 30 μm×50 μm×100 μm skewed rectangular micro-pillars were observed to be the most effective in entrapping the hepatocytes and this design was subsequently used in all future experiments (FIGS. 1B and 2B).

An operating window for the dynamic cell seeding process was also determined. Using real-time fluorescence nuclear staining with Propidium iodide (PI) (Molecular Probes, Oregon) by video imaging, we have validated that cell necrosis post-seeding is highly dependent on the loading flow rate (data not shown). Hepatocytes were introduced into the cell culture device by either infusing or withdrawing a cell suspension with a syringe pump at different flow rates. The minimal achievable flow rate by infusing the cell suspension was 0.5 ml/hr, which was higher than that by withdrawing the cell suspension i.e. 0.1 ml/hr. The mean cell viability was correspondingly higher when hepatocytes were seeded at the minimal flow rate by withdrawing the cell suspension than by infusing the cell suspension (FIG. 2). Therefore, dynamic seeding of the hepatocytes was carried out by withdrawing the cell suspension from a reservoir at the minimal permissible flow rate for a micro-channel of a particular dimension to minimize detrimental effects on the hepatocytes.

### EXAMPLE 3

Modulation of Cell-Matrix Interaction by Different Flow Configurations During Laminar flow Complex Coacervation of Methylated Collagen and HEMA-MMA-MAA Terpolymer.

In this example, it was demonstrated that and extracellular matrix (ECM) can be introduced to the 3-D construct (i.e., cell culture device) independently of the cell localization process using the projections of the cell culture device. In addition, ECM can be modulated to control cell-matrix interactions without affecting the mechanical stability of the 3-D cell construct.

Formation of 3-D Matrix Support for Hepatocytes by Laminar Flow Complex Coacervation

Upon the dynamic seeding of hepatocytes within the cell culture device, a 3-D collagen matrix was formed around the cells by a complex coacervation reaction between a positively charges methylated collagen and a negatively charged HEMA-MMA-MAA terpolymer [Chia et al., 2000]. The 3-D matrices were localized within the cell retention chamber of the cell culture device by virtue of the laminar flow profile within the cell culture device, thereby preventing turbulence mixing between the collagen and terpolymer streams [Toh et al., 2005]. Hepatocytes were re-suspended in 1.5 mg/ml me thylated collagen and dynamically loaded into the cell retention chamber as described in example 2. A 3% terpolymer solution was then infused via the side channels to initiate the complex coacervation reaction (FIGS. 5 and 6). The complex coacervation reaction between methylated collagen and terpolymer was carried out with 2 flow configurations to modulate the degree of gelation of the methylated collagen. In the first configuration, methylated collagen flow was minimized by locking the cell reservoir with a luer lock. In the second configuration, the cell reservoir was left opened to maximize the methylated collagen stream flow as a result of hydrostatic pressure. The terpolymer solution was infused using a syringe pump at 0.1 ml/min for 1 minutes followed by 0.5 ml/ml for 5 minutes. Subsequently, the excess terpolymer solution was removed by perfusing with 1×PBS.

Visualization of Complex Coacervated Collagen Matrices with Confocal Laser Scanning Microscopy (CLSM)

Methylated collagen was labeled with a fluorescence probe, Alexa-Fluor 532 (Molecular Probes, Oregon), and diluted to 1.5 mg/ml with 1×PBS. The 3-D matrix support for hepatocytes after dynamic seeding into the cell retention chamber of a cell culture device (200 μm (W)×100 μm (H)×1 cm (L)) was formed as described above with the 2 flow configurations using the labeled methylated collagen. The nuclei of the hepatocytes were counter-stained by perfusing with 250 nM of Sytox Green (Molecular Probes, Oregon) at 0.8 ml/hr for 30 minutes. The samples were then fixed with 3.7% PFA for minutes before visualization with a confocal microscope (Olympus Fluoview 300).

Visualization of Complex Coacervated Collagen Matrices with Scanning Electron Microscopy (SEM)

SEM samples of the complex coacervated 3-D matrices in the micro-fluidic channels were prepared by preparing the samples immediately after plasma oxidation of the PDMS membrane so that bonding between the PDMS cell culture device and the glass coverslip was not permanent. The samples were fixed by perfusing with 3.7% PFA for 30 minutes and the PDMS cell culture device was peeled off the glass coverslip. The PDMS cell culture device was then post-fixed with 1% osmium tetraoxide for 2 hours, and then sequentially dehydrated by incubating with 25%, 50%, 75%, 95% and 100% ethanol (10 minutes each). The cell culture device was then cut into 5 mm thick cross-sections with a surgical blade and subsequently dehydrated in liquid carbon dioxide. The samples were viewed with JEOL JSM-7400F (JEOL Ltd, Japan).

Results

The degree of cell-matrix interactions between hepatocytes and the 3-D complex coacervated collagen matrices can be modulated by controlling the extent of the complex coacervation reaction. This control of exerted by varying the methylated collagen stream as described by the 2 flow configurations. When flow of the methylated collagen stream is minimal as implemented in configuration 1, the amount of methylated collagen that can complex coacervate with the terpolymer solution was limited, resulting in a conformal layer of collagen fibres surrounding the hepatocytes (data not shown). With an increasing methylated collagen flow as implemented in configuration 2, the amount of material available for complex coacervation with terpolymer increased, forming a fibrous matrix where hepatocytes were embedded in (data not shown). The collagen stream can potentially be regulated to further fine-tune the degree of complex coacervation reaction, thereby controlling the extent of cell-matrix interactions.

The observations of the SEM samples of the 3-D matrices formed within the micro-fluidic channel using configuration 1 corroborated with the observations made using the fluorescence-labeled collagen. Hepatocytes were packed at high density within the micro-pillar array and covered with a thin fibrous shell of coacervated collagen matrix (data not shown).

### EXAMPLE 4

Evaluation of Hepatocytes' Viability after 3-D Seeding Into a Cell Culture Device with Projections and Laminar Flow Complex Coacervation

Primary rat hepatocytes were first three-dimensionally. localized by using the proposed cell culture device with projections, followed by the construction of a 3-D matrix using laminar flow complex coacervation of methylated collagen and HEMA-MMA-MAA terpolymer solution [Toh et al., 2005]. The viability of the hepatocytes was subsequently assessed by fluorescence staining after seeding into the described 3-D patterned construct.

1.5×10⁶ cells/ml of primary rat hepatocytes were suspended in 1.5 mg/ml of methylated collagen and seeded into a micro-channel (200 μm (W)×100 μm (H)×1 cm (L)) by withdrawing at three different flow rates from the cell reservoir, ranging from 0.1-0.02 ml/hr. Following cell seeding, a 3-D matrix was formed around the cell aggregate within the micro-pillar array by the complex coacervation of methylated collagen and HEMA-MMA-MAA terpolymer streams using configuration 1 as described above. After the construction of the 3-D microenvironment of the hepatocytes within the micro-channel, where there were adequate cell-cell and cell-matrix interactions, the viability of the hepatocytes were assessed to investigate the effect of the seeding process according to methodology used in example 2.

Results

Cell viability was negatively correlated to higher withdrawal flow rate as previously reported in example 1 (data not shown). The cell viability at 0.1 ml/hr withdrawal rate was 61.9%, which was significantly lower than the cell viability when a withdrawal rate of 0.05 ml/hr or 0.02 ml/hr was used (>80%). The formation of the 3-D matrix by the complex coacervation did not appear to have detrimental effects on cell viability as cell viability of more than 80% was attainable when the minimal withdrawal flow rate was used. This was consistent with the reported viability achievable without matrix formation in example 2.

### EXAMPLE 5

Perfusion Culture of Bone Marrow Stromal Cells (BMSCs) After 3-D Seeding into a Cell Culture Device with Projections and Laminar Flow Complex Coacervation.

In the following example, the proposed cell culture device with projections was used to three-dimensionally trapped bone marrow stromal cells (BMSCs). The BMSCs in the micro-channel were maintained under perfusion culture for 1 day before assessment of the cell morphology.

Isolation and Culture of Rat Bone Marrow Stromal Cells (BMSCs).

Aspirates of rat bone marrow were plated on T-25 culture flasks and maintained in a 37° C. CO₂ incubator for 24 hours to allow for stromal cells attachment. The bone marrow was then removed and the attached BMSCs were washed 3× with 1×PBS. The BMSCs were then cultured using Dulbecco's modified Eagle medium (DMEM), low glucose (Gibco, Grand Island, N.Y.) supplemented with 10% fetal bovine serum (FBS) and penicillin/streptomycin. The cultures were cultured to about 80% confluence before passaging. Passage 2-7 cells were used in all experiments.

Seeding of Rat BMSCs Into Micro-Fluidic Channel Using Micro-Pillar Array and Laminar Flow Complex Coacervation

5×10⁶ cells/ml of rat BMSCs (P₂) were suspended in 1.5 mg/ml of methylated collagen and seeded into a micro-channel (200 μm (W)×100 μm (H)×1 cm (L)) by withdrawing at flow rate of 0.03 ml/hr from the cell reservoir. Following cell seeding, a 3-D matrix was formed around the cell aggregate within the cell retention chamber by the complex coacervation of methylated collagen and HEMA-MMA-MAA terpolymer streams using configuration 1 described above.

Perfusion Culture of Rat BMSCs in Micro-Fluidic Channel

A closed loop perfusion culture system was set up as shown in FIG. 8. CO₂ independent culture medium consisting of Dulbecco's modified Eagle medium (DMEM), low glucose (Gibco, Grand Island, N.Y.) supplemented with 10% fetal bovine serum (FBS), penicillin/streptomycin and 60 mM HEPES was circulated at a flow rate of 5 μl/min for 24 hours. The micro-channel was placed onto a microscope heating stage to maintain its temperature at 37° C. throughout the culture period.

Results

Cells loaded three-dimensionally in a micro-fluidic channel were able to successfully trap rat BMSCs using the above described conditions. Laminar flow complex coacervated collagen matrices was incorporated independently to stabilize the 3-D cell construct within the micro-channel (data not shown). After 24 hours of perfusion culture, it was observed that the rat BMSCs contracted into a tight 3-D aggregate spanning the length of the cell culture device. Cellular extensions from the aggregate were observed to anchor the aggregate to the projections as well as the walls of the cell culture device (data not shown). The cellular morphology of BMSCs cultured in this proposed 3-D micro-scale in vitro model was distinctively different from BMSCs cultured in 2-D substrates indicating the importance of the dimensionality of the cellular microenvironment (data not shown).

### REFERENCES

Baret F. Isolation, purification and cultivation of rat liver sinusoidal endothelial cells (LSEC). Laboratory Investigation (1994); 70: 944-952.
Chia et al., Hepatocyte encapsulation for enhanced cellular functions. Tissue Engineering (2000); 32: 481-495.
Chiu et al., Patterned deposition of cells and proteins onto surfaces by using three-dimensional microfluidic systems. PNAS (2000); 97(6): 2408-2413.
Toh et al., Complex coacervating microfluidics for immobilization of cells within micropatterened devices. Assay and Drug Development Technologies (2005); 3(2): 162-167.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

The following examples are offered by way of illustration and not by way of limitation.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this invention belongs.

1. A cell culture device comprising a channel, the channel comprising one or more inlets and one or more outlets, and a cell retention chamber defined by an internal surface of the channel and a plurality of projections extending therefrom, wherein the projections are spaced apart at least part of the way along the longitudinal axis of the channel, and wherein the projections are spaced to permit the passage of liquid but not cells between adjacent projections.

2. The cell culture device of claim 1 wherein the channel comprises a bottom wall and side walls.

3. The cell culture device of claim 2 further comprising a top wall.

4. The cell culture device of claim 2 wherein said projections project upwardly from said bottom wall of the channel.

5. The cell culture device of claim 1 wherein the channel has at least two inlets.

6. The cell culture device of claim 5 wherein the channel has at least three inlets.

7. The cell culture device of claim 5 wherein the channel has at least 2 outlets.

8. The cell culture device of claim 5 wherein the channel has at least three outlets.

9. The cell culture device of claim 1 wherein the projections are separated by about 1 to 20 μm.

10-12. (canceled)

13. The cell culture device of claim 1 wherein the projections are circular, semi-circular, rectangular or square.

14-27. (canceled)

28. The cell culture device of claim 1 further comprising means for heating a cell culture contained in the cell retention chamber.

29-32. (canceled)

33. The cell culture device of claim 1, further comprising means for introducing liquid medium.

34-40. (canceled)

41. The cell culture device of claim 1, wherein the cell retention chamber comprises one or more cell cultures.

42. The cell culture device of claim 41 wherein the one or more cell cultures are seeded in the device by laminar flow.

43. (canceled)

44. The cell culture device of claim 41, wherein the one or more cell cultures are embedded in a collagen gel within the chamber.

45-53. (canceled)

54. The cell culture device of claim 1, further comprising a cell reservoir connected to the channel.

55-58. (canceled)

59. The cell culture device of claim 1 in which the cell retention chamber is arranged such that a space is provided for the perfusion of a liquid medium through the channel, the space being defined by a side wall of the channel and a row of the projections.

60. The cell culture device of claim 1, in which the cell retention chamber is arranged such that a space is provided on either side of the chamber for the perfusion of a liquid medium through the channel, each space being defined by a side wall of the channel and a row of the projections.

61-62. (canceled)

63. A method of making the cell culture device of claim 1, said method comprising the steps of:
(a) fabricating a mould using photolithography; and
replica moulding using a polymeric compound.

64-68. (canceled)

69. A method of culturing cells in the cell culture device of claim 1, the method comprising the steps of:
(c) introducing one or more types of cells suspended in methylated collagen into the cell retention chamber of the cell culture device; and
(d) introducing a terpolymer solution to initiate a complex coacervation reaction which results in gradual gelation of the collagen matrix.

70. (canceled)

71. A method for observing a cell culture in a cell culture device of claim 1 for bioimaging comprising:
(a) seeding the cell culture device with one or more cell types in a collagen matrix; and
(b) observing the one or more cell types with an imaging device.

72-75. (canceled)

76. A method of screening a plurality of candidate pharmaceutical compounds against a target comprising:
(a) seeding a plurality of cell culture devices of claim 1 with one or more cell types containing the target in a collagen matrix;
(b) perfusing the cell culture devices with the candidate pharmaceutical compounds; and
(c) screening the cell culture devices to identify the desired compound.

77. A method for purification of a biological fluid comprising:
(a) seeding a plurality of the cell culture devices of claim 1 with one or more cell types in a collagen matrix;
(b) perfusing the cell culture devices with the biological fluid; and
(c) obtaining purified biological fluid.

78-79. (canceled)

80. A method comprising culturing cells in the cell culture device of claim 1.

81. The method of claim 80 wherein the cells are anchorage-dependent cells.

82. The method of claim 81 wherein the cells are selected from the group consisting of hepatocytes, fibroblasts, bone marrow stromal cells, endothelial cells, chondrocytes, osteoblasts, myocytes, neural cells, and stellate cells.

83. The method of claim 82 wherein the endothelial cells are liver sinusoidal endothelial cells (SECs).
